(19) United States
(12) Patent Application Publication
(10) Pub. No.: US 2006/0116534 A1
(43) Pub. Date: Jun. 1, 2006

(54) Process for manufacture of an allyl ether

(76) Inventors: Jesper Samuelsson, Tyringe (SE); Oleg Pajalic, Kristianstad (SE); Mircea Manea, Vittsjo (SE); Keith Ogemark, Porstorp (SE)

Correspondence Address: STEVENS DAVIS MILLER & MOSHER, LLP, 1615 L STREET, NW, SUITE 850, WASHINGTON, DC, 20036, US

(21) Appl. No.: 10/549,611

(22) PCT Filed: Mar. 18, 2004
(86) PCT No.: PCT/SE04/00399

§ 371 Date: Oct. 20, 2005

(30) Foreign Application Priority Data
Mar. 21, 2003 (SE) 0300765-5

(51) Int. Cl. C07C 41/08

(52) U.S. Cl. 568/673

(57) ABSTRACT

A process for production of an allyl and/or methallyl ether of a tri or polyhydric alcohol is disclosed. Said process comprises (i) subjected at least one cyclic formal of a tri or polyhydric alcohol to allylation, (ii) subjecting obtained allyl and/or methallyl ether of said cyclic formal to reaction with at least one alcohol and optionally an intermediate and/or a final purification step. In further aspects the present invention refers to an allyl and/or methallyl ether yielded in said process.

The present invention relates to the production of an allyl and/or methallyl ether of a tri or polyhydric alcohol, such as a 2-alkyl-2-hydroxyalkyl-1,3-propanediol or a 2,2-dihydroxyalkyl-1,3-propanediol and/or a cyclic formal thereof. More specifically, the invention relates to a novel process for the preparation of an allyl and/or methallyl ether from a waste stream comprising at least one cyclic formal of a said tri or polyhydric alcohol. In further aspects the present invention refers to an allyl and/or methallyl ether yielded in said process.

Allyl and methallyl ethers of polyhydric alcohols are typically produced according to the Williamson synthesis by reacting a polyhydric alcohol with at least one allyl or methallyl halide, such as allyl bromide or methallyl chloride, in the presence of a basic catalyst.

Tri and polyalcohols having for instance a neopentyl structure, such as trimethylolpropane, trimethylolethane and pentaerythritol are normally synthesised in an alkali catalysed aldolcondensation of formaldehyde and a second aldehyde. Yielded aldolaldehyde is subsequently reduced to corresponding alcohol, by means of a so called Cannizzaro reaction, with a further amount of formaldehyde in the presence of a strong base. The reaction can alternatively be carried out by means of catalytic hydration. The synthesis as well as recovery of obtained reaction product normally yield secondary products, such as formals (formaldehyde acetals). The syntheses yield linear and cyclic formals, such as 1,3-dioxolanes and 1,3-dioxanes. Linear formals are when exposed to acidic treatment, for instance when passing an ion exchanger, during recovery of the primary product transformed into cyclic formals.

Cyclic formals are also yielded during recovery if a synthesised tri or polyalcohol under acidic conditions is in contact with formaldehyde. This occurs for instance during an evaporation procedure, wherein water and excess of formaldehyde are evaporated. Formation of formals occurs when a 1,3-diol structure in a di, tri or polyalcohol reacts with for instance formaldehyde to corresponding 1,3-dioxane. Glycerol can similarly yield formals, such as 4-hydroxymethyl-1,3-dioxolane and 5-hydroxy-1,3-dioxane. Cyclic formals are furthermore yielded during other polyalcohol syntheses, such as acid catalysed etherification, as disclosed in for instance the U.S. Pat. No. 3,673,226.

Formals yielded as by-products in a synthesis of a polyhydric alcohol are continuously subjected to development efforts making said formals technically and commercially valuable. One such achievement is represented by 5-ethyl-5-hydroxymethyl-1,3-dioxane (cyclic trimethylolpropane formal) now frequently used for instance as flow additive in cement compositions and for preparation of reactive acrylic diluents.

The present invention quite unexpectedly provides a process wherein cyclic formals, for instance yielded as by-products in a synthesis of a tri or polyhydric alcohol, are converted to technically and commercially valuable products, such as allyl ethers of tri and polyhydric alcohols or allyl ethers of said cyclic formals. The present invention accordingly refers to a process for production of an allyl and/or methallyl ether of a tri or polyhydric alcohol.

The process of the present invention comprises the steps of
i) subjecting at least one cyclic formal of a tri or polyhydric alcohol to allylation by reaction with at least one allyl and/or methallyl halide in the presence of a catalytically effective amount of at least one basic catalyst, whereby a reaction mnixture comprising at least one allyl and/or methallyl ether of said cyclic formal is yielded, and
ii) subjecting in step (i) obtained allyl and/or methallyl ether of said cyclic formal to reaction with at least one alcohol, having one or more hydroxyl groups, optionally in presence of a catalytically effective amount of at least one organic acid catalyst, whereby a reaction mixture comprising at least one allyl and/or methallyl ether of said tri or polyalcohol and at least one formal of said alcohol,
   and optionally an intermediate purification step wherein the reaction mixture obtained in step (i) is purified prior to initiation of step (ii) and/or a final purification step wherein the reaction mixture obtained in step (ii) is purified.

Step (i) of said process is preferably performed at atmospheric or at a reduced or increased pressure and at a temperature of 60-140° C. and step (ii) is likewise preferably performed at a reduced pressure, such as a pressure of less than 15 m=Hg, or at a atmospheric or increased pressure and at a temperature of 80-160° C. Said allyl and/or methallyl halide and said basic catalyst are in step (i) advantageously added continuously to the reactor.

Said optional intermediate purification step comprises, in preferred embodiments of the process of the present invention, at least one extraction, such as mixing the reaction mixture obtained in step (i) with water and allowing obtained mixture to separate in a water phase and an organic phase and recovery of said organic phase. Said optional intermediate purification can also and optionally comprise further purifying, of for instance said organic phase, by for instance evaporation, such as distillation, at a pressure of for instance less than 15 mm Hg.

Said optional final purification step comprises, in preferred embodiments of the process of the present invention, purification of the reaction mixture obtained in step (ii) by for instance evaporation, such as distillation, at a pressure of for instance less than 15 mm Hg. Said optional final purification may also comprise extraction as for instance disclosed above for said optional intermediate purification.

The cyclic formal subjected to allylation is suitably a recovered and optionally purified by-product or is present in a mixture, such as a waste stream which optionally is purified, of by-products from a plant wherein a tri or polyalcohol, such as glycerol, trimethylolethane, trimethylolpropane or pentaerythritol, is synthesised and produced.

Preferred embodiments of said cyclic formal include cyclic formals having a 1,3-dioxolane and most preferably a 1,3-dioxane structure, such as cyclic formals of 1,2,3-propanetriols, 2-alkyl-2-hydroxyalkyl-1,3-propanediols, 2-alkyl-2-hydroxyalkoxy-1,3-propandiols, 2-alkyl-2-hydroxyalkoxyalkyl-1,3-propanediols, 2,2-dihydroxyalkyl-1,3-propanediols, 2,2-di-hydroxyalkoxy-1,3-propanediols, 2,2-dihydroxyalkoxyalkyl-1,3-propanediols and dimers, trimers and polymers thereof. Said cyclic formal can suitably be exemplified by a cyclic formal of glycerol, trimethylolethane, trimethylolpropane, diglycerol, ditrimethylolethane, ditrimethylolpropane, pentaerythritol, dipentaerythritol and ethoxylated and/or propoxylated species thereof. Preferred embodiments of said cyclic formal include 4-hydroxyalkyl-1,3-dioxolanes, 5-hydroxy-1,3-dioxanes, 5-alkyl-5-hydroxy-1,3-dioxanes, 5-alkyl-5-hydroxyalkyl-1,3-dioxanes and/or 5,5-hydroxyalkyl-1,3-dioxanes. The most preferred cyclic formals are cyclic formals of tri or polyhydric alcohols having a neopentyl structure, such 2,2-substituted 1,3-propanediols. Especially preferred embodiments of the present invention include subjecting for instance 4-hydroxyalkyl-1,3-dioxolane, 5-hydroxy-1,3-dioxane, 5-alkyl-5-hydroxy-1,3-dioxane, 5-alkyl-5-hydroxyalkyl-1,3-dioxane and 5,5-hydroxyalkyl-1,3-dioxane to allylation in step (i) and subjecting obtained allyl and/or methallyl ether to reaction in step (ii).

Said at least one allyl and/or methallyl halide is, in embodiments of the present invention, preferably allyl and/or methallyl bromide and/or chloride and said at least one basic catalyst is suitably and preferably at least one alkali and/or alkaline earth metal hydroxide, alkoxide and/or carbonate, such as potassium and/or sodium hydroxide, carbonate and/or methoxide.

Said at least one alcohol, having one or more hydroxyl groups, in step (ii) subjected to reaction with the product obtained in step (i) is most preferably at least one mono, di, tri or polyalcohol, such as at least one alkanol, alkanediol, 2,2-alkyl-1,3-propanediol, 2-alkyl-2-hydroxyalkyl-1,3-propanediol, 2,2-dihydroxyalkyl-1,3-propanediol or at least one dimer, trimer or polymer of a said alcohol. Embodiments of said preferred alcohols include the most preferred species, which include methanol, 2-ethylhexanediol, ethylene glycol, neopentyl glycol, trimethylolpropane and trimethylolethane.

Said at least one optional organic acid catalyst is most preferably p-toluenesulphonic acid and/or methanesulphonic acid.

The most preferred embodiments of the process of the present invention include embodiments wherein said cyclic formal subjected to allylation in step (i) is 5,5-dihydroxymethyl-1,3-dioxane optionally present in a mixture, such as a said waste stream, of by-products as disclosed above and that said alcohol in step (ii) subjected to reaction with in step (i) yielded allyl and/or methallyl ether is methanol or trimethylolpropane.

In a further aspect the present invention refers to an allyl and/or methallyl ether of a tri or polyhydric alcohol, which allyl and/or methallyl ether is obtained by the process disclosed above. Said allyl and/or methallyl ether is in the most preferred embodiments a monoallyl, diallyl, monomethallyl and/or dimethallyl ether of pentaerythritol and most preferably obtained from 5,5-dihydroxymethyl-1,3-dioxane recovered from or present in a mixture of by-products, such as a waste stream, yielded in a pentaerythritol synthesis.

In yet a further aspect the present invention refers to a novel allyl and/or methallyl ether yielded in step (i) of the process according to the present invention. Said allyl and/or methallyl ether is most preferably a monoallyl, diallyl, monomethallyl and/or dimethallyl ether of 5,5-dihydroxymethyl-1,3-dioxane, which dioxane is yielded as by-product in a pentaerythritol synthesis.

Without further elaboration, it is believed that one skilled in the art can, using the preceding description, utilise the present invention to its fullest extent. The following preferred specific embodiments are, therefore, to be construed as merely illustrative and not limitative of the remainder of the disclosure in any way whatsoever. In the following Examples 1-4 show embodiments of step (i) of the present process, Examples 5 and 6 show embodiments of step (ii) of the present process, Example 7 shown an embodiment of the optional intermediate purification step and Example 8 shows an embodiment of the optional final purification step.

### EXAMPLE 1

A mixture of polyhydric compounds (Polyol PX™, Perstorp Specialty Chemicals AB, Sweden) obtained from a pentaerythritol synthesis was in an amount comprising 853 g (4 moles) of the cyclic pentaerythritol formal 5,5-hydroxymethyl-1,3-dioxane subjected to reaction with 309 g (4 moles) of allyl chloride in the presence of 352 g (4.4 moles) of sodium hydroxide. The reaction was performed in a 2000 ml 3-necked reaction flask equipped with a thermometer, condenser, Dean-Stark receiver, stirrer and a drop funnel. The reaction was under stirring carried out at a temperature of 120° C. The reaction time was 7 hours. Yielded reaction water was continuously collected in the Dean-Stark receiver. Obtained reaction mixture was neutralised with hydrochloric acid to a pH of 7. Volatile by-products were removed by evaporation at 60° C. and 10 mm Hg and formed sodium chloride was removed by filtration.

877 g of the reaction mixture was recovered and GC analyses showed that a 61% selectivity of monallyl ether of cyclic pentaerythritol formal was obtained with a 56% conversion of cyclic pentaerythritol formal.

### EXAMPLE 2

Example 1 was repeated with the difference that Polyol PX™ in an amount comprising 326 g (1.53 mole) of CPF was subjected to reaction with 296 g (3.83 moles) of allyl chloride and 337 g (4.21 moles) of sodium hydroxide.

390 g of the reaction mixture was recovered and GC analyses showed that a 50% selectivity of monallyl ether of cyclic pentaerythritol formal was obtained with a 100% conversion of cyclic pentaerythritol formal.

### EXAMPLE 3

Example 1 was repeated with the difference that Polyol PX™ in an amount comprising 759 g (4.97 moles) of CPF was subjected to reaction with 384 g (4.97 moles) of allyl chloride and 437 g (5.46 moles) of sodium hydroxide.

895 g of the reaction mixture was recovered and GC analyses showed that a 82% selectivity of monallyl ether of cyclic pentaerythritol formal was obtained with a 72% conversion of cyclic pentaerythritol formal.

### EXAMPLE 4

Example 1 was repeated with the difference that Polyol PX™ in an amount comprising 1128 g (7.4 moles) of CPF was subjected to reaction with 687 g (8.88 moles) of allyl chloride and 710 g (8.88 moles) of sodium hydroxide.

1426 g of the reaction mixture was recovered and GC analyses showed that a 87% selectivity of monallyl ether of cyclic pentaerythritol formal was obtained with a 88% conversion of cyclic pentaerythritol formal.

### EXAMPLE 5

400 g of a reaction mixture, comprising 1.32 mole of monoallyl ether of cyclic pentaerythritol formal, obtained in accordance with Examples 1-4, was subjected to reaction with 385 g (2.81 moles) of trimethylolpropane in the presence of 4.9 g (0.05 mole) of p-toluenesulphonic acid. The reaction was performed in a 2000 ml 3-necked reaction flask equipped with a thermometer, stirrer and a distillation column with a Sulzer packing. The reaction was under stirring carried out at a temperature of 135° C. and a pressure of 1 mm Hg. The reaction time was 6 hours.

1219 g of reaction mixture was recovered and GC analyses showed that a 95% selectivity of pentaerythritol monoallyl ether was obtained with a 87% conversion of monoallyl ether of cyclic pentaerythritol formal.

### EXAMPLE 6

1500 g of a reaction mixture, comprising 7.91 moles of monoallyl ether of cyclic pentaerythritol formal, obtained in accordance with Examples 1-4, was subjected to reaction with 2105 g (65.7 moles) of methanol in the presence of 15.0 g (0.079 mole) of p-toluenesulphonic acid. The reaction was performed in a 3-necked reaction flask equipped with a thermometer, stirrer and a distillation column with a Sulzer packing was. The reaction was under stirring carried out at a temperature of 85° C. and a pressure of 760 mm Hg. The reaction time was 72 hours.

### EXAMPLE 7

1893 g of a reaction admixture, obtained in accordance with Examples 1-4, comprising 93.3% monoallyl ether and 5.3% diallyl ether of cyclic pentaerythritol formal was purified in a batch distillation equipment at a pressure of 1 mm Hg. The distillation column was packed with a Sulzer BX packing. The reflux was 10:1 at the beginning of the distillation and 5:1 at the end.

1298 g of the main fraction was recovered. The distillation yield was 73% and GC analyses showed a 99% purity of monoallyl ether of cyclic pentaerythritol formal.

### EXAMPLE 8

1398 g of a reaction admixture, obtained in accordance with Examples 5 and 6, comprising 89.9% pentaerythritol monoallyl ether was purified in a batch distillation equipment at a pressure of 1 mm Hg. The column was packed with a Sulzer BX packing. The reflux was 10:1.

1113 g of the main fraction was recovered. The distillation yield was 87% and GC analyses showed 98% purity of pentaerythritol monoallyl ether.

1. A process for production of an allyl and/or methallyl ether of a tri or polyhydric alcohol wherein said process comprises the steps of:
i) subjecting at least one cyclic formal of at least one tri or polyhydric alcohol to allylation by reaction with at least one allyl and/or methallyl halide in presence of a catalytically effective amount of at least one basic catalyst, whereby a reaction mixture, comprising at least one allyl and/or methallyl ether of said cyclic formal, is yielded, and
ii) subjecting in step (i) yielded allyl and/or methallyl ether of said cyclic formal to reaction with at least one alcohol, having one or more hydroxyl groups, optionally in presence of a catalytically effective amount of at least one organic acid catalyst, whereby a reaction mixture, comprising at least one allyl and/or methallyl ether of said tri or polyhydric alcohol and at least one formal of said alcohol, is yielded.

2. A process according to claim 1, wherein said step (i) is performed at a temperature of 60-140° C.

3. A process according to claim 1, wherein said step (ii) is performed at a temperature of 80-160° C.

4. A process according to claim 1, wherein said optional intermediate purification comprises extraction and optionally further purification by evaporation.

5. A process according to claim 1, wherein said optional final purification step comprises purification of the reaction mixture obtained in step (ii) by evaporation.

6. A process according to claim 1, wherein said at least one cyclic formal is a recovered by-product or is present in a mixture of by-products from a synthesis of a tri or polyalcohol.

7. A process according to claim 1, wherein said at least one cyclic formal is recovered from a waste stream and/or a mixture of by-products from a synthesis of a tri or polyalcohol and optionally that said cyclic formal is purified.

8. A process according to claim 1, wherein said at least one cyclic formal is at least one cyclic formal selected from the group consisting of a 1,2,3-propanetriol, 2-alkyl-2-hydroxyalkyl-1,3-propanediol, 2-alkyl-2-hydroxyalkoxy-1,3-propandiol, 2-alkyl-2-hydroxyalkoxyalkyl-1,3-propanediol, 2,2-dihydroxyalkyl-1,3-propanediol, 2,2-dihydroxyalkoxy-1,3-propanediol or 2,2-dihydroxyalkoxyalkyl-1,3-propanediol.

9. A process according to claim 1, wherein said at least one cyclic formal is at least one cyclic formal of at least one dimer, trimer or polymer selected from the group consisting of a 1,2,3-propanetriol, 2-alkyl-2-hydroxyalkyl-1,3-propanediol, 2-alkyl-2-hydroxyalkoxy-1,3-propandiol, 2-alkyl-2-hydroxyalkoxyalkyl-1,3-propanediol, 2,2-dihydroxyalkyl-1,3-propanediol, 2,2-dihydroxyalkoxy-1,3-propanediol or 2,2-dihydroxyalkoxyalkyl-1,3-propanediol.

10. A process according to claim 1, wherein said at least one cyclic formal is at least one cyclic formal selected from the group consisting of glycerol, trimethylolethane, trimethylolpropane, diglycerol, ditrimethylolethane, ditrimethylolpropane, pentaerythritol or dipentaerythritol.

11. A process according to claim 1, wherein said at least one cyclic formal is at least one cyclic formal selected from the group consisting of an ethoxylated and/or propoxylated glycerol, trimethylolethane, trimethlolpropane, diglycerol, ditrimethylolethane, ditrimethylolpropane, pentaerythritol or dipentaerythritol.

12. A process according to claim 1, wherein said at least one cyclic formal is selected from the group consisting of at least one 4-hydroxyalkyl-1,3-dioxolane, 5-hydroxy-1,3-dioxane, 5-alkyl-5-hydroxy-1,3-dioxane, 5-alkyl-5-hydroxyalkyl-1,3-dioxane or 5,5-hydroxy-alkyl-1,3-dioxane.

13. A process according to claim 12, wherein at least one cyclic formal is selected from the group consisting of 5-hydroxy-1,3-dioxane, 5-methyl-5-hydroxymethyl-1,3-dioxane, 5-ethyl-5-hydroxymethyl-1,3-dioxane or 5,5-dihydroxymethyl-1,3-dioxane.

14. A process according to claim 1, wherein said at least one allyl halide is allyl, methallyl bromide or chloride.

15. A process according to claim 1, wherein said at least one basic catalyst is at least one alkali or alkaline earth metal hydroxide, alkoxide or carbonate.

16. A process according to claim 15, wherein said at least one basic catalyst is potassium or sodium hydroxide, carbonate or methoxide.

17. A process according to claim 1, wherein said at least one alcohol, having one or more hydroxyl groups, is at least one mono, di, tri or polyalcohol.

18. A process according to claim 17, wherein said at least one mono, di, tri or polyalcohol is an alkanol, an alkanediol, a 2,2-alkyl-1,3-propanediol, a 2-alkyl-2-hydroxyallkyl-1,3-propanediol, a 2,2-dihydroxyalkyl-1,3-propanediol or a dimer, trimer, or polymer of a said alcohol.

19. A process according to claim 17, wherein said at least one mono, di, tri or polyalcohol is methanol, 2-ethylhexanediol, ethylene glycol, neopentyl glycol, trimethylolpropane and/or trimethylolethane.

20. A process according to claim 1, wherein said at least one organic acid catalyst is p-toluenesulphonic acid or methanesulphonic acid.

21. A process according to claim 1, wherein said at leaset one cyclic formal subjected to allylation in step (i) is 5,5-dihydroxymethyl-1,3-dioxane or a mixture, such as a waste stream, comprising 5,5-dihydroxymethyl-1,3-dioxane and that said alcohol, which in step (ii) is subjected to reaction within step (i) yielded allyl and/or methallyl ether, is trimethylolpropane.

22. An allyl ether of a tri or polyhydric alcohol, wherein it is yielded in the process of claim 1.

23. An allyl and/or methallyl ether according to claim 22, wherein said allyl and/or methallyl ether is at least one monoallyl, dially, monomethallyl and/or dimethallyl ether of pentaerythritol.

24. A novel allyl and/or methallyl ether, wherein it is yielded in step (i) of the process according to claim 1.

25. A novel allyl and/or methallyl ether according to claim 24, wherein said allyl and/or methallyl ether is at least one monoallyl, diallyl, monomethallyl and/or dimethallyl ether of 5,5-dihydroxymethyl-1,3-dioxane.

26. A novel allyl and/or methallyl ether according to claim 25, wherein said 5,5-dihydroxymethyl-1,3-dioxane is yielded as by-product in a synthesis of pentaerythritol.

27. A process according to claim 1, wherein said at least one methallyl halide is allyl, methallyl bromide or chloride.

28. An methallyl ether of a tri or polyhydric alcohol, wherein it is yielded in the process of claim 1.

29. The process according to claim 4, wherein said purification step is distillation.

30. The process according to claim 5, wherein said purification step is distillation.
